(19) United States
(12) Patent Application Publication
(10) Pub. No.: US 2017/0082568 A1
(43) Pub. Date: Mar. 23, 2017

(54) SYSTEM AND METHOD OF SENSING SOIL MOISTURE

(71) Applicant: WaterBit, Inc., Santa Clara, CA (US)

(72) Inventors: Manu Pillai, San Jose, CA (US); Kevin Seichi Yamada, Sunnyvale, CA (US)

(21) Appl. No.: 15/274,914

(22) Filed: Sep. 23, 2016

Related U.S. Application Data
(60) Provisional application No. 62/222,693, filed on Sep. 23, 2015.

(51) Int. Cl. G01N 27/22; G01N 33/24

(52) U.S. Cl. CPC G01N 27/223; G01N 27/228; G01N 33/246

(57) ABSTRACT

A system and method of sensing the amount of moisture in soil are disclosed. According to an embodiment, a soil moisture sensing system comprises a circuit unit and a sensor link electrically connected to the circuit unit. The sensor link includes a plurality of segments, each segment including an LC circuit in which a capacitor is connected with an inductor. The circuit unit is configured to provide an oscillating signal to a first LC circuit of the sensor link, sense a first coupled signal in a second LC circuit of the sensor link, and determine a soil moisture level on the basis of the first coupled signal.

### RELATED APPLICATIONS

This application claims priority to and the benefit of U.S. Provisional Application No. 62/222,693, filed on Sep. 23, 2015, as authorized under 35 U.S.C. §119. The entire contents of this provisional application are incorporated herein by reference.

### RELATED FIELD

The present disclosure relates in general to a system and method of sensing the amount of moisture in soil.

### BACKGROUND

Generally, soil moisture is the water that is trapped in the spaces among soil particles. Determining the amount of soil moisture has important implications for a number of reasons, especially in the science of agriculture. For example, soil moisture serves as a solvent and carrier of food nutrients for plant growth, as well as regulates soil temperature. As such, crop yield is often determined by the amount of water available in the soil rather than the deficiency of other food nutrients.

While soil moisture sensing systems already exist, they all suffer from certain flaws in one way or another due to their measurement technique. Examples of known techniques for measuring soil moisture include: (1) receive signal strength indicator (RSSI)/radio attenuation, (2) electrical permittivity (i.e., a capacitive measure), (3) time-domain reflectometry (TDR), and (4) electrical conductivity (i.e., a resistive measure). (This is not an exhaustive list.)

The RSSI/radio attenuation measurement technique generally involves placing a transmitter above ground and a receiver sensor buried about 10˜15 cm in the soil. However, because the transmitter has to operate with the power guidelines for the respective frequency as per applicable regulations, such measurement technique is limited to turf applications or similar applications where a shallow reading is sufficient. Furthermore, because resulting measurements are affected by changes in the humidity of air, compensations for these effects are needed. Also, because the growth of plants interferes with radio transmission, application of such measurement technique is limited to frequently trimmed/mowed environments where soil moisture measurement is concerned.

The electrical permittivity measurement technique generally involves a fork-like sensor or separated surfaces that enable permittivity measurements between the prongs of the fork-like sensor or between the separated surfaces. AC electrical signals, either high or low frequency, are sent from one prong/surface and received on the other prong/surface. Changes in permittivity, and thus capacitance, of the soil between the two prongs/surfaces impact the attenuation of the signal. The received signal may be rectified using an AC rectifier to generate a DC level. However, a problem with the electrical permittivity measurement technique is that the electric field formed between the two prongs/surfaces is a fringe field, and thus, the center of the prongs/surfaces used is often a “dead zone.” Additionally, the surfaces need to have soil compacted tightly against them, whether directly exposed as a metallic surface, or hidden behind installation materials and packaging in order to work. As such, the prongs/surfaces of such measurement devices are typically short (e.g, 6˜12 cm), their sensing radius is limited and accuracy is heavily dependent on the quality of the soil compaction against the sensing surfaces.

The TDR technique generally involves pulse generation and capture. Probes are typically wired with an external telemetry and computation unit. However, because this technique depends on the effect of signal reflection from the adjacent soil and how the signal is impacted, it suffers from the same issues related to the permittivity measurement technique, as this is essentially a metric for resolving the complex and real vectors of the lumped impedance model of the soil; small changes in soil composition—from compaction density to roots to stones—impact the signal. As a result, this technique is often used in turf and lawn measurements as well, with some application to agriculture. In commercial agriculture and geologic soil surveys, TDR techniques coupled with eddy current measurements have been applied to measurements since the 1980s; however, these techniques require significant separation between transmission and receiver elements, as well as significant power and orientation constraints, rendering them ineffective for large scale commercial agricultural use on a sustained basis.

The electrical conductivity measurement technique generally operates under the assumption that the electrical resistance of the soil changes with its moisture level. However, because the resistance of the soil also changes with salt content in addition to moisture level, false measurements may occur. Furthermore, such measurement technique may be challenging when the soil is loose since it also generally requires direct contact with the soil for electrical conductivity.

In addition to the issues listed above, the sensing systems utilizing the above techniques tend to be highly sensitive to the makeup of the soil directly next to its sensors, and provide measurements that can be highly variable due to issues in installation or other sources. The volume of soil measured may also be an issue, as each of the sensing systems described above measures only a very short distance from its sensor probes (generally just a few centimeters).

In view of the foregoing, there exists a need for a system and method of sensing soil moisture that overcome the drawbacks of traditional instruments and techniques.

### SUMMARY

A system and method of sensing the amount of moisture in soil are disclosed. According to an example embodiment, a soil moisture sensing system comprises a circuit unit and a sensor link electrically connected to the circuit unit. The sensor link includes a plurality of segments, each segment including an LC circuit in which a capacitor is connected with an inductor. The circuit unit is configured to provide an oscillating signal to a first LC circuit of the sensor link, sense a first coupled signal in a second LC circuit of the sensor link, and determine a soil moisture level on the basis of the first coupled signal.

According to an example embodiment, a method of soil moisture sensing utilizes a sensor link having a plurality of segments, each segment including an LC circuit in which a capacitor is connected with an inductor. The method comprises: driving an oscillating signal to a first LC circuit to generate an oscillating magnetic field; detecting a first coupled signal in a second LC circuit; rectifying the first coupled signal and converting the magnitude of the rectified signal into a first digital value; and comparing or matching the first digital value with calibrated data to determine a first soil moisture level corresponding to a first depth.

### BRIEF DESCRIPTION OF THE DRAWINGS

The accompanying drawings, which are included as part of the present disclosure, illustrate various embodiments and together with the general description given above and the detailed description of the various embodiments given below serve to explain and teach the principles described herein.

FIG. 1 illustrates a partially disassembled soil moisture sensing system, according to an exemplary embodiment of the present disclosure.

FIG. 2 illustrates one base segment by itself when it is not linked to form a sensor link, according to an exemplary embodiment of the present disclosure.

FIG. 3 illustrates an equivalent circuit diagram of the LC circuits respectively formed on the base segments, according to an exemplary embodiment of the present disclosure.

FIG. 4 is a flow chart of a process of measuring the moisture content of soil, according to an exemplary embodiment of the present disclosure.

FIG. 5 illustrates an exemplary computer architecture that may be used for the present system and method, for example the recommendation engine.

The figures in the drawings are not necessarily drawn to scale and elements of similar structures or functions are generally represented by like reference numerals for illustrative purposes throughout the figures. The figures are only intended to facilitate the description of the various embodiments described herein and do not describe every aspect of the teachings disclosed herein and do not limit the scope of the present disclosure.

### DETAILED DESCRIPTION

Each of the features and teachings disclosed herein may be utilized separately or in conjunction with other features and teachings to provide a system and method of evaluating the integrity of soil moisture and other in-soil sensors. Representative examples utilizing many of these features and teachings, both separately and in combination, are described with reference to the attached figures. While the detailed description herein illustrates to a person of ordinary skill in the art further details for practicing aspects of the present teachings, it does not limit the scope of the present disclosure. Therefore, combinations of features disclosed in the detailed description are representative examples of the present teachings and may not be necessary to practice the teachings in the broadest sense.

As mentioned above, traditional soil moisture sensing systems have a number of limitations due to the underlying principles and techniques upon which the sensors operate. Disclosed herein is a novel system and method for detecting and measuring soil moisture content that addresses a number of key deficiencies of traditional soil moisture detection systems. According to exemplary embodiments of the present system and method, the effects of a magnetic field, or an “H” field, are used to accurately measure soil moisture content at greater depths, with greater measurement radii, across different soil types, and at different levels of depth.

That is, unlike the traditional soil moisture sensing systems that measure electric fields to determine permittivity or those that measure electric currents to determine conductivity, the present system and method measure the effects of magnetic fields. Particularly, according to embodiments of the present system and method, an oscillating magnetic field is generated in the soil using a transmit coil, and an alternating current (AC) eddy current signal is induced in the nearby soil, depending on the level of moisture, which is then received at a receive coil according to Faraday's Law, as applied to mutual induction. The AC signal in the receive coil varies according to the magnetic coupling of the magnetic field with the eddy current that is induced by and proportional to the soil moisture content, and thus is referred to as a “coupled signal” herein. The coupled signal may be rectified and converted to a digital value for quantification purposes. As the moisture level increases, the coupled signal also increases because moisture increases the eddy current effect, thus increasing the magnetic coupling between coils. The oscillating frequency of the magnetic field generated by the transmit coil may be a mid-range frequency that is above the mains frequency. The mains frequency refers to the frequency of the general household AC current frequency. For example, the mains frequency is 60 Hz in North America and 50 Hz in Europe. By implementing the present system and method with a low power transmitter and receiver, the electronic emissions profile of the measurement system is maintained at a very low level, leading to an implementation that is economic and efficient.

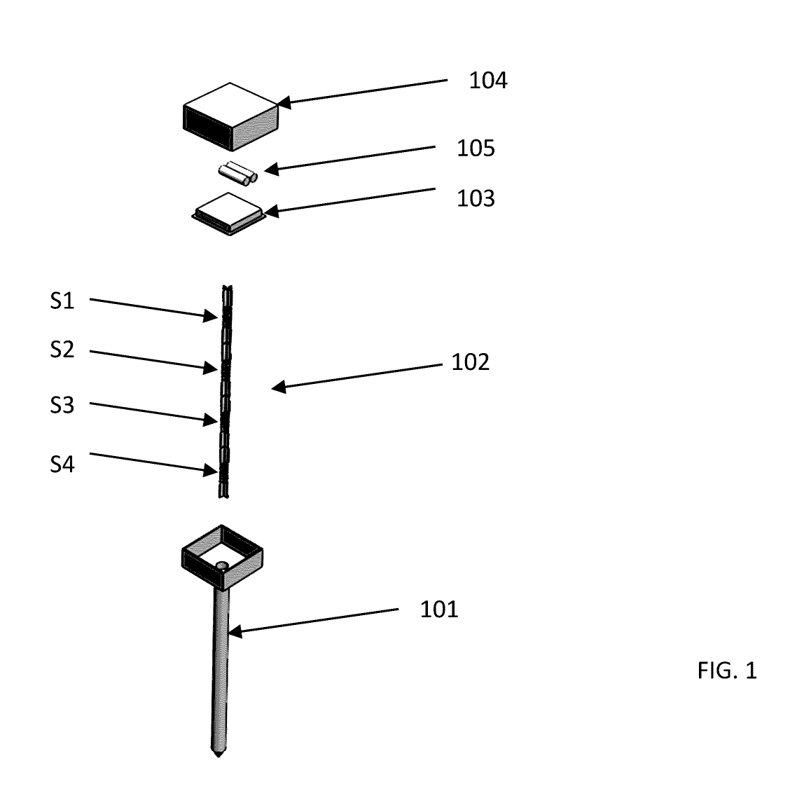

FIG. 1 illustrates a partially disassembled soil moisture sensing system, according to an exemplary embodiment of the present disclosure. The system includes a protective housing 101, a sensor link 102, a main circuit 103, and a battery or power source housing 104, which is referred to herein as a “battery housing”. The battery housing 104 is configured to house and electrically connect one or more batteries/power sources 105 to the main circuit 103 to provide power thereto. The protective housing 101 is configured to encase the sensor link 102 therein to shield and protect the sensor link 102 from environmental hazards, such as moisture and other corrosive elements, when the encased sensor link 102 is inserted into the ground. The main circuit 103 may include, among other components, a wireless transceiver for transmitting and receiving data to and from a wireless base station, another sensing system, or any other wireless device.

The sensor link 102 includes a plurality of base segments S that are configured to be attachable and detachable from each other so that the length of the sensor link 102 may be modularly increased or decreased according to use. FIG. 1 shows four base segments—S1, S2, S3 and S4—attached to each other to form the overall structure of sensor link 102, but the present system and method are not limited thereto. Any number of base segments may be linked together to form the sensor link 102.

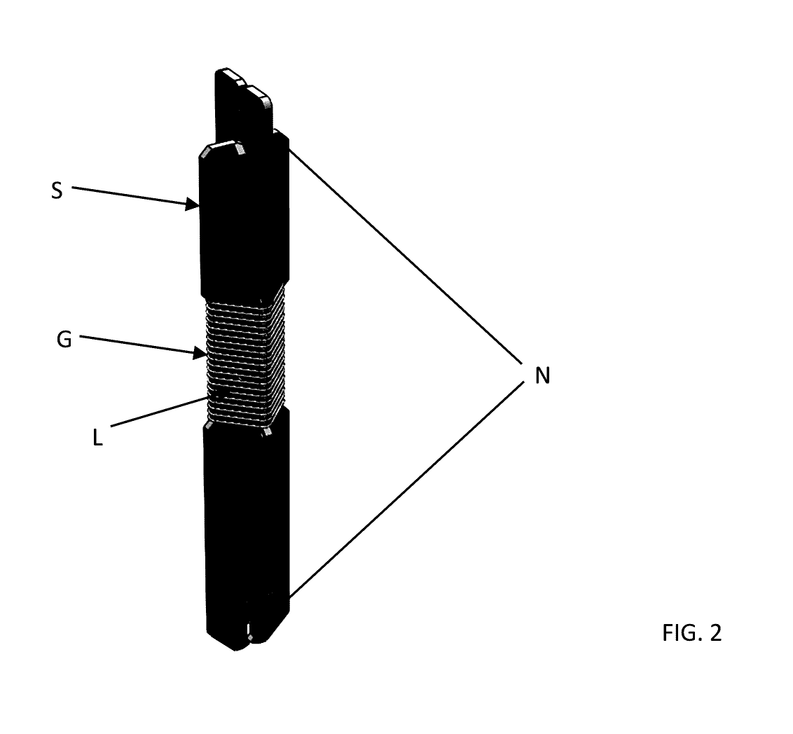

FIG. 2 illustrates one base segment by itself when it is not linked to form the sensor link 102, according to an exemplary embodiment. The base segment S includes a number of fins (four in this case) on which a plurality of grooves G (e.g., sawtooth grooves) are formed. The grooves G are configured to maintain the spacing between adjacent loops of a wire that is wound around the base segment S to form a link of the sensor link 102, which is described further below. On one or more of the fins, holes may be formed to accommodate and hold in place the terminals of a capacitor and/or the wire that are used to form the sensor link, also described further below, in addition to any other circuit elements or mechanical parts specific to an implementation scenario. Also, the ends of the base segment S may include notches or other features N for linking to other base segments.

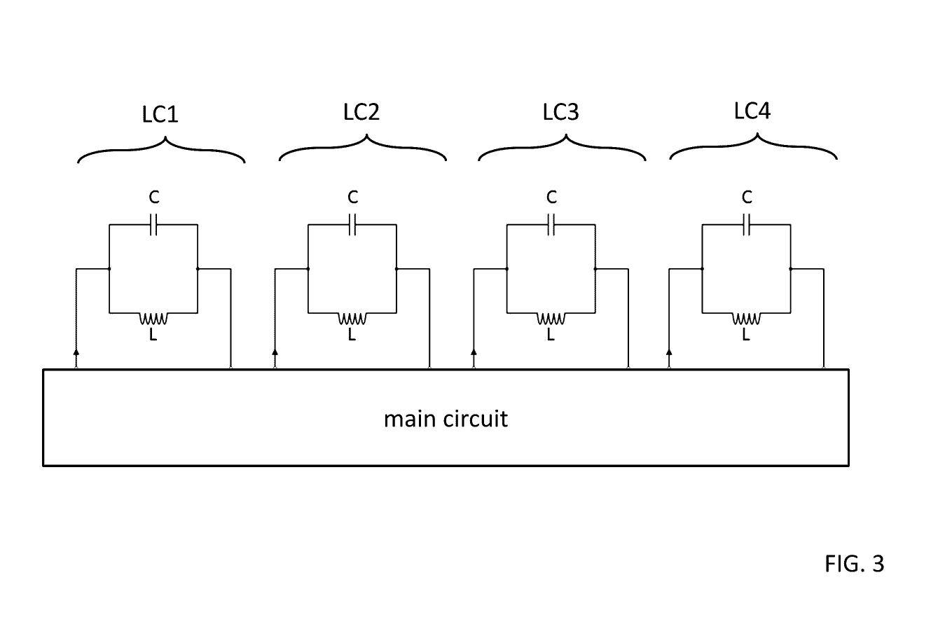

Referring back to FIG. 1, a wire is wrapped around the grooves of each base segment to form a coil, which functions as an inductor L. As such, the terms “coil” and “inductor” are used interchangeably herein. The ends of the wire are respectively connected to the terminals of a capacitor C (not shown in the figures). Thus, for each base segment of the sensor link 102, an inductor L and a capacitor C are connected to each other to form an LC circuit. The inductance values and the capacitive values of the inductor L and capacitor C, respectively, determine the resonant frequency of the LC circuit. FIG. 3 illustrates an equivalent circuit diagram of the LC circuits—LC1, LC2, LC3 and LC4—that are respectively formed on the base segments S1 to S4. The input and output terminals of each LC circuit are connected to the main circuit 103.

As mentioned earlier, soil moisture detection according to exemplary embodiments of the present disclosure involves generating an oscillating magnetic field using a transmit coil and detecting a coupled signal using a receive coil. Here, each of the LC circuits of FIG. 3, or each sensor link, may act as a transmit coil or a receive coil, depending on how the LC circuit is operated by the main circuit 103. For example, the main circuit 103 may drive LC circuit LC1 with an oscillating signal (e.g., Sinusoidal voltage signal), in which case LC circuit LC1 acts as a transmit coil, and listen for a coupled signal using the LC circuit LC2, in which case LC circuit LC2 acts as a receive coil. Similarly, the main circuit 103 may drive LC circuit LC2 with an oscillating signal, in which case LC circuit LC2 acts as a transmit coil, and listen for a coupled signal using the LC circuit LC3, in which case LC circuit LC3 acts as a receive coil.

When the encased sensor link 102 is inserted perpendicularly into a soil bed, the length of the sensor link 102 corresponds roughly to the maximum depth under the soil bed at which soil moisture content can be accurately measured. That is, when positioned in this manner, the coils are oriented in a vertically stacked manner. Thus, by measuring the coupled signal from different receive coils along the length of the sensor link 102, the soil moisture level at different depths (up to the maximum depth) under the soil bed may be detected and measured. In one embodiment, the coils may be arranged in pairs, with the distance separating the coils being the zone in which moisture is detected. In another embodiment, the main circuit 103 may methodically alternate the roles of each LC circuit along the length of the sensor link 102 from one end to the other end (e.g., “walking” the coils) so that a soil moisture depth profile is obtained.

An advantage of the present system and method is that measurements can be accurately performed in loose soil, such as amended soil and rich loamy soil, as well as compacted soil. To illustrate, consider a cylindrical volume of soil surrounding the sensor link 102. By driving a subset number of the coils to transmit (e.g., one coil at a time) for each measurement, the moisture content of the soil can be measured in a piecewise, linear manner that sums up to the cylindrical volume. Moreover, the magnetic field strength F for each coil is defined by a relationship illustrated below:

f(Coil(length,diameter,density),Transmit(power,distance),Moisture(radius,height))

In other words, the magnetic field strength F for each coil is directly related to the transmit power level used, as well as the base moisture level. Thus, by controlling the transmit power in the subset of coils (e.g., one coil) to measure at a particular radius of the cylindrical volume, the radius of the cylindrical volume that can be measured is much higher (˜10 inches) than those measurable by traditional systems, which have been typically around 2 cm, and a moisture profile in 3 dimensions, across time, can be created. An increased radius enables a much better estimate of water content at a given depth. The type of soil may impact the effective radius. In a cost-constrained version, power control can be eliminated, leading a more traditional and simpler moisture profile.

Further, each coil interacts with the transmit coil in such a way that the magnetic fields are formed with each receive coil and each transmit coil. That is, the total magnetic field strength at any specific coil is the sum of how each coil interacts with the transmit coil, assuming no losses

F(N)=Sum(F(1)+F(2) . . . +F(n))

For example, a Field2 on Coil2 is impacted by a Field1, a Field3, and so on. In other words, the total field effect is a cumulative effect. However, by controlling the transmission power level, more discrimination in field overlap can be achieved, while arranging coils in pairs can improve discrimination further, and reducing the effect of overlapping fields.

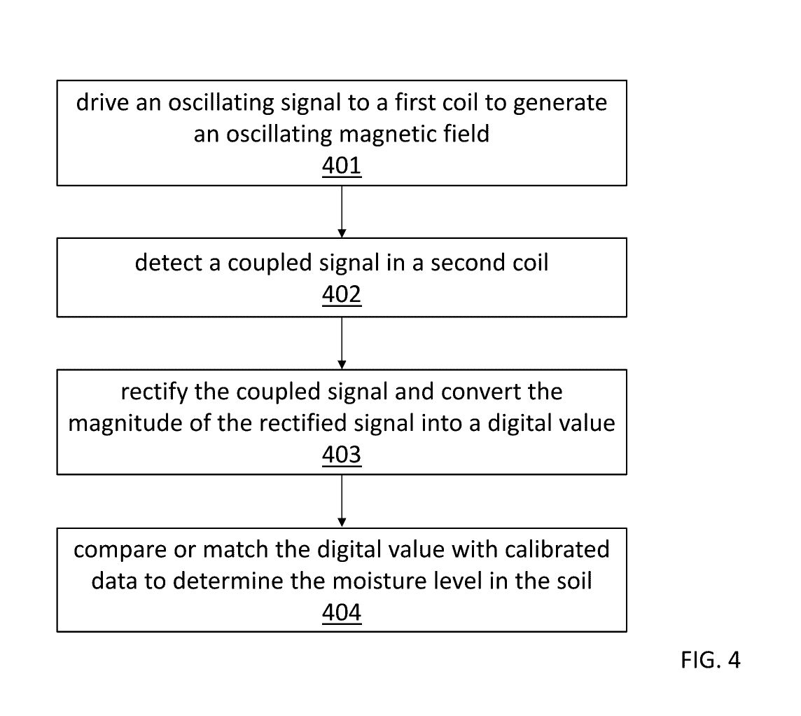

FIG. 4 is a flow chart of a process of measuring the moisture content of soil, according to an exemplary embodiment of the present disclosure. Although the main circuit is described below as performing the process of FIG. 4, those of ordinary skill in the art would understand that other equivalent circuits or components may perform some or all of the process without departing from the teachings herein. At 401, the main circuit drives an oscillating signal to a first coil to generate an oscillating magnetic field. The oscillating signal may be a smoothed clock signal that closely approximates a Sinusoidal signal. In response to the oscillating magnetic field, the moisture in the soil creates an induced magnetic field. At 402, the main circuit detects a coupled signal in a second coil. The coupled signal is an AC signal that is induced in the second coil by the effects of overlapping magnetic fields, which includes the oscillating magnetic field generated by the first coil and the induced magnetic fields generated by the water and soil. At 403, the main circuit rectifies the coupled signal and converts the magnitude of the rectified signal into a digital value. The magnitude of the rectified signal may be a root-means-square (RMS) value. At 404, the main circuit compares or matches the digital value with calibrated data to determine the moisture level in the soil. The calibrated data may be stored in a storage component of the main circuit as a table of signal values and corresponding moisture levels, or the data could also be transmitted to a remote location for further processing. The calibrated data may be predetermined, for example, using gravimetric measurement. Gravimetric measurement involves determining the weight of a volume of soil before and after drying. As noted earlier, the process of FIG. 4 may be repeated with different coils along the length of the sensor link so that soil moisture content may be measured at different soil depths.

In some embodiments, the calibrated data may be retrieved from a database based on the soil type in which the measurement is to be performed. The soil type defines the soil density and composition. The calibrated data may also be retrieved from the database based on the geographical location of the soil to be measured. For example, a smartphone may be used in conjunction with the sensor system of FIG. 1 to identify the location of where the sensor system is to be used, retrieve the corresponding calibrated data from a database, and store the calibrated data in the main circuit.

Another aspect of applying vertically stacked coils in the manner described earlier is to better measure the correlation of water percolation rates, soil types and water tension. Water tension is another key parameter that horticulturists and farmers measure in the soil. It is a measure of how tightly the soil holds onto its water content, which directly correlates to how much work a plant's root system has to do to obtain water. Water tension is generally measured in Pa (pressure) using a tensiometer.

According to exemplary embodiments, the present system and method may also be used to measure water tension. In particular, because the present system and method enable detection of soil moisture level at different soil depths, the rate at which moisture percolates through the soil may be measured, and the gravimetric potential of the water may be derived. By comparing the moisture data against gravimetric data and against time, a regression model for the rate at which water percolates through soil may be derived. Models may be created for various soil types. Furthermore, by applying machine learning techniques that include other metrics such as air temperature, soil type, leaf and/or stem water potential, watering interval and timing, as well as the migration of peak moisture levels after watering, the soil water tension levels may be estimated.

According to exemplary embodiments, the main circuit may implement a power management scheme that matches watering cycles. For example, the main circuit may limit soil moisture measurements and transmission of the measured data to the time periods just before, during and after irrigation, in addition to a few other measurements in between for detection of percolation and root health patterns. Power management may also react to the time of day, days within growth cycle times, predicted irrigation cycle times, as well as user query patterns.

According to exemplary embodiments, the present system and method may be utilized along with a plant-location specific recommendation engine that integrates different data models. In particular, the recommendation engine may integrate location specific data, such as soil type, temperature, wind speed, humidity, evapotranspiration potential, etc., with other data, such as the plant type, season and growth/harvest cycle of the plant, to drive recommendations. These recommendations may range from highly-optimized watering profiles to nutrition to insect/bug prevention that are linked to precision plant models. The recommendation engine may also leverage existing models in the public domain, then apply machine learning techniques to develop models that reflect granularity down to the area of measurement. This could range from a plot to a specific plant.

In summary, the present system and method have many benefits, including but not limited to: (1) the ability to measure moisture level at different depths in the soil, thereby enabling watering only when and where really needed, (2) the ability to model the rate at which water percolates through the soil, and integrate that into watering patterns, (3) the ability to time watering cycles to reduce evaporation losses, (4) the ability to model salt aggregation against deep watering and flush needs against growth cycles. (5) The ability to detect increases in soil moisture, for example as a precursor to damage to buildings

The foregoing description, for purposes of explanation, used specific nomenclature to provide a thorough understanding of the present system and method. However, it will be apparent to one skilled in the art that the specific details are not required in order to practice the present system and method. Thus, the foregoing descriptions of specific embodiments of the present system and method are presented for purposes of illustration and description. They are not intended to be exhaustive or to limit the present system and method to the precise forms disclosed. Many modifications and variations are possible in view of the above teachings. The embodiments were chosen and described in order to best explain the principles of the present system and method and their practical applications, to thereby enable others skilled in the art to best utilize the present system and method and various embodiments with various modifications as are suited to the particular use contemplated. The various features of the disclosed embodiments and examples may be mixed and matched in any manner or combination to form further embodiments contemplated by the present disclosure.

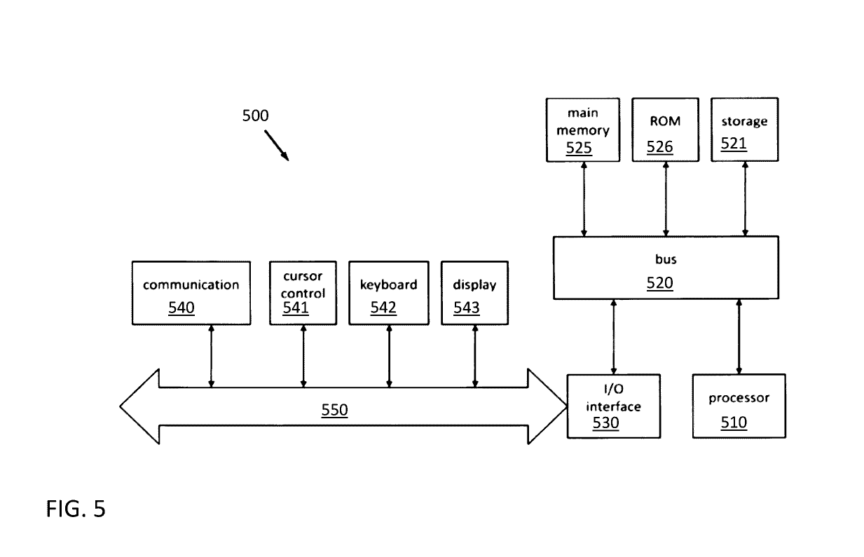

FIG. 5 illustrates an exemplary computer architecture that may be used for implementing the present system and method. The exemplary computer architecture may be used for implementing one or more components described in the present disclosure including, but not limited to, the recommendation engine. One embodiment of architecture 500 comprises a system bus 520 for communicating information, and a processor 510 coupled to bus 520 for processing information. Architecture 500 further comprises a random access memory (RAM) or other dynamic storage device 525 (referred to herein as main memory), coupled to bus 520 for storing information and instructions to be executed by processor 510. Main memory 525 also may be used for storing temporary variables or other intermediate information during execution of instructions by processor 510. Architecture 500 may also include a read only memory (ROM) and/or other static storage device 526 coupled to bus 520 for storing static information and instructions used by processor 510.

A data storage device 521 such as a magnetic disk or optical disc and its corresponding drive may also be coupled to architecture 500 for storing information and instructions. Architecture 500 can also be coupled to a second I/O bus 550 via an I/O interface 530. A plurality of I/O devices may be coupled to I/O bus 550, including a display device 543, an input device (e.g., an alphanumeric input device 542, a cursor control device 541, and/or a touchscreen device).

The communication device 540 allows for access to other computers (e.g., servers or clients) via a network. The communication device 540 may comprise one or more modems, network interface cards, wireless network interfaces or other interface devices, such as those used for coupling to Ethernet, token ring, or other types of networks.

Some portions of the detailed description herein are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the below discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The present disclosure also relates to an apparatus for performing the operations herein. This apparatus may be specially constructed for the required purposes, or it may comprise a general purpose computer selectively activated or reconfigured by a computer program stored in the computer. Such a computer program may be stored in a computer readable storage medium, such as, but is not limited to, any type of disk, including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, and each coupled to a computer system bus.

The algorithms presented herein are not inherently related to any particular computer or other apparatus. Various general purpose systems, message servers, or personal computers may be used with programs in accordance with the teachings herein, or it may prove convenient to construct a more specialized apparatus to perform the required method steps. The required structure for a variety of these systems will appear from the description below. It will be appreciated that a variety of programming languages may be used to implement the teachings of the disclosure as described herein.

Moreover, the various features of the representative examples and the dependent claims may be combined in ways that are not specifically and explicitly enumerated in order to provide additional embodiments of the present teachings. The dimensions and the shapes of the components shown in the figures are designed to help understand how the present teachings are practiced and do limit the dimensions and the shapes shown in the examples.

What is claimed is:

1. A soil moisture sensing system comprising:
a circuit unit; and
a sensor link electrically connected to the circuit unit,
wherein the sensor link includes a plurality of segments, each segment including an LC circuit in which a capacitor is connected with an inductor, and
wherein the circuit unit is configured to:
provide an oscillating signal to a first LC circuit of the sensor link,
sense a first coupled signal in a second LC circuit of the sensor link, and
determine a soil moisture level on the basis of the first coupled signal.

2. The soil moisture sensing system of claim 1, wherein the circuit unit is further configured to:
provide an oscillating signal to the second LC circuit of the sensor link,
sense a second coupled signal in the first LC circuit of the sensor link, and
determine a soil moisture level on the basis of the second coupled signal.

3. The soil moisture sensing system of claim 1, wherein the circuit unit includes a wireless transceiver configured to communicate with a wireless base station or another soil moisture sensing system.

4. The soil moisture sensing system of claim 1, wherein each segment of the sensor link includes a plurality of fins.

5. The soil moisture sensing system of claim 4, wherein one or more of the fins includes a plurality of uniformly spaced grooves.

6. The soil moisture sensing system of claim 5, wherein each segment of the sensor link includes a wire wrapped around the uniformly spaced grooves to form a coil that serves as the inductor of the LC circuit.

7. The soil moisture sensing system of claim 6, wherein each segment of the sensor link includes a notch for linking to another segment of the sensor link.

8. The soil moisture sensing system of claim 1, wherein determining a soil moisture level on the basis of the first coupled signal includes:
rectifying the first coupled signal and converting the magnitude of the rectified signal into a digital value,
comparing or matching the digital value with calibrated data to determine the moisture level in the soil.

9. The soil moisture sensing system of claim 1, further comprising a protective housing configured to encase the sensor link.

10. The soil moisture sensing system of claim 1, wherein the protective housing has a cylindrical shape.

11. The soil moisture sensing system of claim 1, wherein the circuit unit is further configured to implement a power management scheme according to irrigation cycles.

12. A method of soil moisture sensing utilizing a sensor link having a plurality of segments, each segment including an LC circuit in which a capacitor is connected with an inductor, the method comprising:
driving an oscillating signal to a first LC circuit to generate an oscillating magnetic field;
detecting a first coupled signal in a second LC circuit;
rectifying the first coupled signal and converting the magnitude of the rectified signal into a first digital value; and
comparing or matching the first digital value with calibrated data to determine a first soil moisture level corresponding to a first depth.

13. The method of soil moisture sensing of claim 12, further comprising:
driving an oscillating signal to the second LC circuit to generate an oscillating magnetic field;
detecting a second coupled signal in a third LC circuit;
rectifying the second coupled signal and converting the magnitude of the rectified signal into a second digital value; and
comparing or matching the second digital value with calibrated data to determine a second soil moisture level corresponding to a second depth.

14. The method of soil moisture sensing of claim 13, further comprising:
generating a soil moisture depth profile using the first soil moisture level and the second soil moisture level.
